(19) United States
(12) Patent Application Publication
(10) Pub. No.: US 2014/0335176 A1
(43) Pub. Date: Nov. 13, 2014

(54) DISINTEGRANT-FREE DELAYED RELEASE DOXYLAMINE AND PYRIDOXINE FORMULATION AND PROCESS OF MANUFACTURING

(71) Applicant: Pharmascience Inc., Montreal (CA)

(72) Inventors: Prashant Monohar Mandaogade, Pointe-Claire (CA); David Ma, Brossard (CA); Naresh Talwar, Kirkland (CA)

(21) Appl. No.: 14/363,694

(22) PCT Filed: Dec. 7, 2012
(86) PCT No.: PCT/CA2012/001128

§ 371 Date: Jun. 6, 2014

(30) Foreign Application Priority Data
Dec. 7, 2011 (CA) 2761212

(51) Int. Cl. A61K 9/28; A61K 31/4415; A61K 31/4402

(52) U.S. Cl. CPC A61K 9/28; A61K 31/4402; A61K 31/4415; A61K 9/2846
USPC 424/465; 514/357; 514/345

(57) ABSTRACT

The present invention relates to a delayed release pharmaceutical composition containing doxylamine succinate and pyridoxine HCl for treatment of nausea and vomiting during pregnancy. More specifically, the present invention concerns a disintegrant-free delayed release pharmaceutical composition for oral administration comprising a core and an enteric coating, wherein said core comprising: a) at least one pharmaceutically active ingredient, and b) at least one pharmaceutically acceptable excipient, wherein said composition provides an in vitro drug release profile of about 80% of active ingredient dissolved within 20 minutes as measured by USP Type II apparatus and also a manufacturing process of said pharmaceutical composition.

### FIELD OF THE INVENTION

The present invention relates to a delayed release pharmaceutical composition containing multiple active ingredients. More specifically, the present invention is directed to pharmaceutical formulations containing doxylamine succinate and pyridoxine hydrochloride as the active ingredients in a disintegrant-free formulation and processes for manufacturing same.

### BACKGROUND OF THE INVENTION

A number of pharmaceutical dosage forms comprise multiple active ingredients. One example is pharmaceutical compositions containing doxylamine succinate and pyridoxine HCl. This anti-nausea medicament used during pregnancy is well-known in the prior art and is currently sold in Canada by Duchesnay Inc. under the trademark Diclectin®.

A known formulation of Diclectin comprises the following active ingredients: pyridoxine HCl and doxylamine succinate, as the active ingredients, and the following excipients: lactose, microcrystalline cellulose, magnesium trisilicate, silicon dioxide and magnesium stearate. The formulation is sugar coated and suffers from drawbacks, one of which being its delayed onset of action.

Canadian Patent No. 2,350,195 (issued Jun. 6, 2003 to Duchesnay) discloses a formulation containing enterically-coated doxylamine succinate and pyridoxine HCl in a “rapid onset” formulation comprising the following non-active excipients: a filler or binder, a disintegrating agent, a lubricant, a silica flow conditioner and a stabilizing agent.

Another patent, Canadian Patent No. 2,406,592 (issued Sep. 30, 2003 to Duchesnay), discloses the process for preparing pharmaceutical dosage forms comprising multiple active ingredients, namely doxylamine succinate and pyridoxine HCl. The method comprises the steps of mixing said active ingredients and at least one chosen excipient so as to obtain a powdered mixture; compacting said powdered mixture in a roller-compactor apparatus to obtain a compacted product; breaking and sieving said compacted product to a chosen mesh size to obtain similar sized granules; preferably dry mixing said granules with at least on chosen excipient so as to obtain a granular mixture; forming said granular mixture into unitary dosage forms.

Such known formulations have a few drawbacks. The product calls for both active ingredients to be present in reasonably equal amounts. These active ingredients are obtained in the form of powders having different granular sizes which makes it very difficult to uniformly mix them in a dry state along with the required excipients. This can, at times, pose a content uniformity challenge during manufacturing of final dosage forms.

It has been stated that the granulated solid compositions of the existing formulation can be improved by augmenting their dispersibility, i.e. by including a disintegrant such as croscarmellose sodium in the granulation).

There are three most common methods of tablet preparation: (1) direct compression or tabletting; (2) dry granulation; and (3) wet granulation. In direct compression, the powdered material to be included in the tablet (including the active ingredients and excipients) are blended together and compressed directly without intermediate processing such as granulation.

Because direct compression requires fewer unit operations than wet granulation, it is a less expensive process. This means less equipment, lower power consumption, less space, less time, and less labor, all of which reduces the production cost of tablets. However, direct compression is limited to those situations where the compression mix has the requisite physical characteristics required for formation of a pharmaceutically acceptable tablet. Because the tablet formulation is compressed to prepare the tablet, the formulation must possess physical characteristics that lend themselves to processing in such a manner. Among other things, the tablet formulation must be free-flowing, must be lubricated, and, importantly, must possess sufficient binding to ensure that the tablet remains intact after compression.

Disintegrants constitute an important part of the formulation of tablets and capsules of poorly soluble, fluffy and sticky drugs. A disintegrant facilitates break-up or disintegration of a tablet into particles after administration. A significant influence of different formulation components was observed on the tablet dissolution and disintegration with the filler and disintegrating agent exerting the most significant influence. At constant filler or disintegrating agent, an increase in disintegration time led to slower tablet dissolution.

Disintegrants expand and dissolve when wet causing the tablet to break apart in the digestive tract, releasing the active ingredients for absorption. They ensure that when the tablet is in contact with water, it rapidly breaks down into smaller fragments, facilitating dissolution. Examples of disintegrants include: crosslinked polymers, crosslinked polyvinylpyrrolidone (crospovidone), crosslinked sodium carboxymethyl cellulose (croscarmellose sodium), the modified starch sodium starch glycolate, etc.

A drug given in an orally administered tablet must undergo dissolution before it can be absorbed and transported into the systemic circulation. For many drug, dissolution must be preceded by disintegration of tablet matrix. Far tablet dissolution it is necessary to overcome the cohesive strength introduced in to the mass by compression. Therefore, usual practice to incorporate a disintegrant will induce this process.

Disintegration is frequently considered a prerequisite for drug dissolution, however, it in no manner assures that the drug will sufficiently dissolve and have the potential for satisfactory bioavailability. Therefore it is important to assess the effectiveness of the disintegrant on the rate of dissolution of the drug in a tablet (Gissinger D, Stamm A, “A comparative study of cross-linked carboxy-methylcellulose as a tablet disintegrant.” Pharm Ind, 1980; 42: 189-92.)

However the use of disintegrants has a few disadvantages, for example:
not all effective disintegrants swell in contact with water; and
starch-based disintegrants and cellulose derivatives may result in the increase of viscosity after disintegration;

Further, it is known that tablets containing 10% disintegrant must be protected from atmospheric moisture because storage at 60-70% relative humidity may lead to softening of the tablets.

Furthermore, direct compression as a method of tablet manufacture puts many of the traditional disintegrants at a disadvantage due to:
1) high concentrations needed for optimum disintegrating efficiency;
2) poor disintegration in insoluble systems;
3) susceptibility to high compression forces which decreases the efficiency of a disintegrant;
4) poor compression properties; and
5) decreased disintegration efficiency in hydrophobic formulations (Andries, F M, Mingna S and De Villiers M M, “Effect of compression force, humidity and disintegrant concentration on the disintegration and dissolution of directly compressed furosemide tablets using croscarmellose sodium as disintegrant,” Trop. J. Pharma. Res. 2003; 2 (1):125-135).

Also any addition of other excipients (i.e. disintegrant) further leads to an increase in the cost of the dosage form.

Good binding and disintegration properties are obtained with microcrystalline cellulose when it is used in direct compression tablet formulations. However, the material flow properties are relatively poor for most grades of microcrystalline cellulose. Intermittent and non-uniform flow can occur as the formulations move from the hopper to the die on a tablet press. Sometimes microcrystalline cellulose can also have lubricant sensitivity that refers to the reduction in bonding between the plastically-deforming particles in the powder due to the addition of lubricant, which leads to reduction in tablet strength or hardness. Lubricant sensitivity is the ratio of the unlubricated compactability of the tablet formulation to the lubricated compactability of the tablet formulation.

Microcrystalline cellulose (MCC) despite being considered as one of the best dry binders possesses poor flow and disintegration properties and shows capping problems, especially when used in high amounts. Thus, there is considerable interest among pharmaceutical scientists involved in this area of research to either modify the existing products or develop new materials with properties that satisfy as many requirements as possible for direct compression (Swarbrick and Boylan, 2002).

In the development of a solid oral dosage form, the choice of the core excipients is extremely important. Several aspects of the finished dosage form must be considered, such as the nature of the active pharmaceutical ingredients (API), the intended delivery method of the API (e.g, immediate or delayed release), and the manufacturing process.

Various types of formulations to improve the efficacy of drugs comprising doxylamine succinate and pyridoxine HCl have been developed to increase patient compliance, such as women suffering from nausea and vomiting of pregnancy (NVP), who require relief of symptoms.

Thus, it is still desirable to provide patients suffering from nausea and vomiting improved formulations and methods of manufacturing for overcoming the drawbacks of the prior art.

### SUMMARY OF THE INVENTION

The present invention provides a disintegrant-free delayed release doxylamine succinate and pyridoxine HCl formulation and a manufacturing process by using direct compression or dry granulation, which is simple and less expensive. Also, there is provided a formulation exhibiting similar dissolution curves for both active ingredients so as to avoid the dissolution of one active ingredient to the detriment of the other. The therapeutic effect of the active ingredients in said disintegrant-free delayed release formulation is substantially the same as that provided by Diclectin®.

The present invention further provides a disintegrant-free pharmaceutical composition of doxylamine succinate and pyridoxine HCl prepared by direct compression with mannitol and dibasic calcium phosphate as the diluent-filler. The use of disintegrant-free delayed release formulation results in less expensive and simple formulation, with greater physical stability of coated tablets containing the active ingredients at elevated humidity and temperatures.

One aspect of the present invention provides for a disintegrant-free delayed release pharmaceutical composition for oral administration comprising a core and an enteric coating, wherein said core comprises:
a) at least one pharmaceutically active ingredient and
b) at least one pharmaceutically acceptable excipient, wherein said composition provides an in vitro drug release profile of about 95% of the active ingredient dissolved within 20 minutes as measured by USP Type II apparatus, at 100 rpm in 900 ml at pH 6.5 phosphate buffer.

Preferably, the pharmaceutically active ingredient consists of doxylamine succinate, pyridoxine hydrochloride or a combination thereof. More preferably, said pharmaceutical composition comprising 10 mg of doxylamine succinate and 10 mg of pyridoxine hydrochloride.

Also preferably, said composition provides an in vitro drug release profile of about 95% of active ingredient dissolved within 20 minutes as measured by USP Type II apparatus, at 100 rpm in 900 ml at pH 7.5 phosphate buffer.

More preferably, said composition provides an in vitro drug release profile of about 95% of active ingredient dissolved within 140 minutes as measured by USP Type II apparatus, at 100 rpm in change-over Media (for 2 hours in 900 ml of 0.1N HCl and then 1 hour in 900 ml at pH 65 phosphate buffer or pH 75 phosphate buffer).

A further aspect of the present invention provides a disintegrant-free delayed release pharmaceutical composition for oral administration comprising doxylamine succinate and pyridoxine HCl along with at least one pharmaceutically acceptable excipient, and which is substantially free of lactose, wherein an in vitro dissolution profile of said composition provides of about 80% of the each active ingredient dissolved within 20 minutes, as measured by USP Type II Apparatus at 100 rpm in 900 ml at pH 6.5 phosphate buffer.

Preferably, said disintegrant-free delayed release pharmaceutical composition comprising doxylamine succinate and pyridoxine HCl along with at least one pharmaceutically acceptable excipient, and which is substantially free of lactose, wherein an in vitro dissolution profile of said composition provides of about 80% of the each active ingredient dissolved within 20 minutes, as measured by USP Type II Apparatus at 100 rpm in 900 ml at pH 7.5 phosphate buffer.

More preferably, an in vitro dissolution profile of said composition provides about 80% of the each active ingredient dissolved within 140 minutes, as measured by USP Type II Apparatus at 100 rpm in change-over media (for 2 hours in 900 ml of 0.1N HCl and then 1 hour in 900 ml at pH 6.5 phosphate buffer or pH 7.5 phosphate buffer).

Another aspect of the present invention provides a disintegrant-free delayed release pharmaceutical composition for oral administration comprising: (a) doxylamine succinate and pyridoxine HCl as the active pharmaceutical ingredients, (b) mannitol as the filler-diluent, (c) dibasic calcium phosphate dihydrate as a (d) hypromellose as a binder, (e) magnesium stearate as a lubricant, and (f) acrylic enteric polymer coating.

Preferably, the tablet is enteric coated to provide delayed drug release and additional stability to the dosage form. The enteric coated tablets can be printed using Opadry Pink®, or any other suitable colourant. Said formulation is substantially free of lactose, microcrystalline cellulose, sodium croscarmelose and other such disintegrants.

Preferably, the delayed release pharmaceutical composition comprises doxylamine succinate and pyridoxine HCl along with at least one pharmaceutically acceptable excipient selected from the group consisting of: binders, fillers, diluents, hydrophilic polymers, lubricants, glidants, surfactants, coating polymers and combinations thereof.

Preferably, said core comprises at least one pharmaceutically active ingredient, at least one filler; at least one diluent; at least one binder; and at least one lubricant.

Preferably, a disintegrant-free delayed release pharmaceutical composition for oral administration is a tablet, wherein said pharmaceutical composition further comprises at least one enteric coating.

Another aspect of present invention provides a disintegrant-free delayed release pharmaceutical composition for oral administration comprising:
a) at least one pharmaceutically active ingredient;
b) at least one filler;
c) at least one binder;
d) at least one lubricant; and
e) at least one enteric coating, wherein said composition provides an in vitro drug release profile of about 95% of active ingredient dissolved within 20 minutes as measured by USP Type II apparatus, at 100 rpm in 900 ml at pH 6.5 phosphate buffer.

Preferably, said composition provides an in vitro drug release profile of about 95% of active ingredient dissolved within 20 minutes as measured by USP Type II apparatus, at 100 rpm in 900 ml at pH 7.5 phosphate buffer.

More preferably said composition provides an in vitro drug release profile of about 95% of active ingredient dissolved within 140 minutes as measured by USP Type II apparatus, at 100 rpm in change-over Media (for 2 hours in 900 ml of 0.1N HCl and then 1 hour in 900 ml at pH 6.5 phosphate buffer or pH 7.5 phosphate buffer).

Yet another aspect of present invention provides a process for manufacture of a disintegrant-free delayed release pharmaceutical composition for oral administration of claim 1, comprising a core and an enteric coating, wherein said core comprising:
a) at least one pharmaceutically active ingredient;
b) at least one filler;

c) at least one binder; and
e) at least one lubricant, and
   said enteric coating comprises:
f) an aqueous acrylic enteric system;
g) an Antifoam®; and
h) an Opacode®,
wherein said composition provides an in vitro drug release profile of at least 80% of active ingredient dissolved within 20 minutes as measured by USP Type II apparatus, at 100 rpm in 900 ml at pH 6.5 phosphate buffer.

Preferably, said composition provides an in vitro drug release profile of about 90% of active ingredient dissolved within 20 minutes as measured by USP Type II apparatus, at 100 rpm in 900 ml at pH 7.5 phosphate buffer.

More preferably, said composition provides an in vitro drug release profile of at least 80% of active ingredient dissolved within 140 minutes as measured by USP Type II apparatus, at 100 rpm in change-over Media (for 2 hours in 900 ml of 0.111 HQ and then 1 hour in 900 ml at pH 6.5 phosphate buffer or at pH 7.5 phosphate buffer.

Preferably, disintegrant-free delayed release pharmaceutical composition is prepared by direct compression or dry granulation.

More preferably, a process for manufacture of a disintegrant-free delayed release pharmaceutical composition is direct compression and comprises following steps:
a) mixing hypromellose, doxylamine succinate, pyridoxine HQ and a portion of mannitol in a suitable blender;
b) passing the mixture of step (a) through a Comil equipped with a 0.024″R sieve at low speed with a round impeller;
c) mixing mixture of step (b) and another portion of mannitol in a suitable blender;
   c) passing the mixture of step (c) through a Comil equipped with a 0.032″R sieve at low speed with a round impeller;
   d) mixing mixture of step (d) and dibasic calcium phosphate dehydrate, and the rest of the mannitol remaining in the formulation in a suitable blender;
   e) passing mixture of step (e) through a Comil equipped with a 0.032″R sieve at low speed with a round impeller;
   f) re-introducing the blend from step (f) in a suitable bin blender, for 12 minutes at 14 rpm;
   g) mixing magnesium stearate and a small portion of the blend of step (g) and disperse for 30 seconds;
   h) passing the blend of step (h) through a Comil equipped with a 0.018″R sieve at low speed with a round impeller;
   i) incorporating the sieved mixture from step (i) with the rest of the blend from step (g) in a suitable bin blender and mixing for 3 minutes at 14 rpm;
   j) compressing obtained blend in step (j) using rotary tablet press;
   k) coating obtained core tablets with Acryl-eze® and Antifoam®1520 coating dispersion, and
   l) optionally on each coated tablet is printed “P” logo with Opacode® Pink S-1-14022.

The present invention is further related to use of a therapeutically effective amount of a disintegrant-free delayed release pharmaceutical composition comprising doxylamine succinate and pyridoxine HCl for treatment of nausea and vomiting during pregnancy, but not limiting to that.

Preferably, the use of doxylamine succinate and pyridoxine HCl in pharmaceutical composition to prepare a medicament to treat nausea and vomiting and a condition which can benefit from administration of said medicament, wherein said disintegrant-free medicament provides an in vitro dissolution profile of about 80% of the each active ingredient dissolved within 15 minutes, which is substantially the same as provided by Diclectin®.

### DETAILED DESCRIPTION OF THE INVENTION

The present invention relates to a disintegrant-free delayed release pharmaceutical composition for oral administration comprising multiple active ingredients formulation, using a direct compression process which allows to get an delayed release dosage form for doxylamine succinate and pyridoxine HCl, which is used for the treatment of nausea and vomiting during pregnancy, but not limiting to that.

The term “delayed release pharmaceutical composition”, as referred to herein, is defined to mean oral pharmaceutical compositions which, when administered, releases the active ingredient at a time later than that immediately following its administration and provides plasma concentrations of the active ingredient with time within the therapeutic range of the active ingredient over a 24-hour period and encompasses “prolonged release”, “extended release”, “modified release”, “delayed release” and “sustained release” compositions.

Enteric/delayed release coatings consist of pH sensitive polymers, which means the coating remains intact in the acidic environment of the stomach and then solubilizes in the more alkaline environment of the small intestine. Enteric protection for solid oral dosage forms is required to prevent gastric mucosal irritation, to protect a drug which is unstable in gastric fluids or to delay release for local delivery in the intestine. A fully formulated, one-step, dry acrylic enteric coating system dispersible in water for the application of an enteric/delayed release coating to solid dosage forms such as beads, tablets and granules.

The term “active ingredient” refers to an Active Pharmaceutical Ingredients (API) which are active chemicals used in the manufacturing of drugs. The active agent can be a therapeutic, a prophylactic, or a diagnostic agent.

The term “therapeutically effective amount” intends to describe an amount of the active agent which stops or reduces the progress of the condition to be treated or which otherwise completely or partly cures or acts palliative on the condition.

Drug release and drug release profiles are measures or representations of the manner and timing by which a formulation releases or delivers active ingredients (drug) to a receiving environment (e.g. the stomach, intestines, etc.) upon administration. Various methods are known for evaluating drug release and producing release profiles, including in vitro tests which model the in vivo behavior of a formulation. These include USP dissolution testing for immediate release and controlled release solid dosage forms.

The term “Intestinal release systems” means that a drug may be enteric coated for intestinal release for several known reasons such as to prevent gastric irritation, prevent destabilization in gastric pH etc.

“Direct compression” is the simplest and most economical method for the manufacturing of tablets because it requires less processing steps than other techniques such as wet granulation and roller compaction.

Direct compression is the simplest technique to prepare matrix tablets. The matrix system has several advantages as follows: it is very simple and easy to establish a formulation; the tablet is completely dissolved and thus achieves good bioavailability; it is easy to control the dissolution profile by selecting a specific grade; the matrix system is an economical method for obtaining controlled release products.

All formulation components i.e., filler, binder, disintegrating agents, lubricant etc were found to influence tablet dissolution and disintegration, with the filler and disintegrating agent exerting the most significant influence.

“Disintegrating agent” accelerates tablet disintegration into smaller fragments increasing the surface area exposing to the medium for dissolution of the drug to occur. The results highlight the importance and influence of other formulation components, e.g., filler, binder, etc., on the dissolution process and cautions against relying solely on the disintegrating agent to accelerate tablet dissolution.

In a direct compression process, drug is blended with a variety of excipients, subsequently lubricated and directly compressed into a tablet. A “disintegrant” used in this type of formulation, simply has to break the tablet apart to expose the drug substance for dissolution. The ability to interact strongly with water is essential to disintegrant function.

The terms “disintegrant free” and “disintegrant-free” as referred to herein means the pharmaceutical composition is substantially free of disintegrants, such as microcrystalline cellulose, sodium croscarmelose, and other disintegrants known in the art (for example, see the discussion of disintegrants in those defined in Remington: The Science and Practice of Pharmacy (20th edition, 2000)).

The term “filler and diluents” as referred to herein, are defined to mean components that are incorporated into tablet or capsule dosage forms to increase dosage form volume or weight. Sometimes referred to as fillers, diluents often comprise a significant proportion of the dosage form, and the quantity and type of diluent selected often depends on its physical and chemical properties. Fillers fill out the size of a tablet or capsule, making it practical to produce and convenient for the consumer to use. By increasing the bulk volume, the fillers make it possible for the final product to have the proper volume for patient handling. Good filler must be inert, compatible with the other components of the formulation, non-hygroscopic, relatively cheap, compactible, and preferably tasteless or pleasant tasting.

According to present invention a filler-diluent is “mannitol” which is water soluble, non-hygroscopic and produces a semi-sweet, smooth, cool taste. It can be advantageously combined with other direct compression excipients. Amongst the currently available excipients, mannitol provides certain unique advantages.

According to present invention the filler is “dicalcium phosphate dihydrate”, which is the most common inorganic salt used in direct compression as filler. Advantage of using dicalcium phosphate in tablets for vitamin and mineral supplement is the high calcium and phosphorous content. Dicalcium phosphate dihydrate is slightly alkaline with a pH of 7.0 to 7.4, which precludes its use with active ingredients that are sensitive to even small amount of alkali.

The term “binder” as referred to herein, is defined to be incorporated into formulations to hold the ingredients in a tablet together. Binders ensure that tablets and granules can be formed with required mechanical strength, and give volume to low active dose tablets.

According to present invention the binder is hypromellose, which is hydroxypropyl methyl cellulose with a very low viscosity. HPMC including good flow, compressibility, minimal segregation tendency, and good physical and chemical compatibility combined with the ability to provide controlled-drug release.

The term “lubricant” as referred to herein, is defined to be incorporated into formulations to reduce the frictional forces between particles and between particles and metal contact surfaces of manufacturing equipment such as tablet punches and dies used in the manufacture of solid dosage forms. According to present invention the lubricant is Magnesium stearate.

The term “Acryl-EZE® Aqueous Acrylic Enteric System” as referred to herein, is defined to a fully formulated, one-step, dry acrylic enteric coating system dispersible in water for the application of an enteric/delayed release coating to solid dosage forms such as beads, tablets and granules.

The “coloring agent” is incorporated into dosage forms in order to produce a distinctive appearance that may serve to differentiate a particular formulation from others that have a similar physical appearance.

According to present invention, the delayed release is achieved by disintegrant-free composition comprising a core and an enteric coating, wherein said core comprising:
a) at least one pharmaceutically active ingredient, and
b) at least one pharmaceutically acceptable excipient,
wherein said composition provides an in vitro drug release profile of about 95% of active ingredient dissolved within 20 minutes as measured by USP Type II apparatus, at 100 rpm in 900 ml at pH 6.5 phosphate buffer.

Preferably, said composition provides an in vitro drug release profile of about 95% of active ingredient dissolved within 20 minutes as measured by USP Type II apparatus, at 100 rpm in 900 ml at pH 7.5 phosphate buffer.

More preferably, said composition provides an in vitro drug release profile of about 95% of active ingredient dissolved within 1.40 minutes as measured by USP Type II apparatus, at 100 rpm in change-over Media (for 2 hours in 900 ml of 0.1N HCl and then 1 hour in 900 ml at pH 6.5 phosphate buffer or pH 7.5 phosphate buffer.

The present invention provides an advantage for preparing a delayed release disintegrant-free formulation of doxylamine succinate and pyridoxine HCl tablets by direct compression which provides a delayed release dosage form which is used for the treatment of nausea and vomiting during pregnancy.

Furthermore, the present invention particularly provides a more conventional manufacturing process, which is less time consuming, is very simple and can be easily transferred to commercial manufacturing.

The pharmaceutical composition according to the present invention comprises a core comprising:
a) at least one pharmaceutically active ingredient;
b) at least one filler;
c) at least one binder; and
d) at least one lubricant,
   and an enteric coating which envelops the core, the entire coating comprising:
e) an Acryl-eze® (White 93O18359);
f) an Atifoam®; and
g) a colorant Opacode®.

Preferably, the pharmaceutically active ingredient of said pharmaceutical composition consists of doxylamine succinate, pyridoxine hydrochloride or a combination thereof.

More preferably, said composition comprises 10 mg of doxylamine succinate and 10 mg of pyridoxine hydrochloride.

The pharmaceutical composition according to the present invention comprising doxylamine succinate and pyridoxine HO along with at least one pharmaceutically acceptable excipient. Said composition is disintegrant-free and provides the in vitro dissolution profile of at least about 80% of each active ingredient dissolved within 20 minutes, as measured by LISP Type II Apparatus at 100 rpm in 900 ml at pH 6.5 phosphate buffer.

Preferably, said composition provides the in vitro dissolution profile of at least 80% of doxylamine succinate and at least 80% of pyridoxine HCl dissolved within 20 minutes, as measured by USP Type II Apparatus at 100 rpm in 900 ml at pH 7.5 phosphate buffer.

More preferably, said composition provides the in vitro dissolution profile of at least about 80% of doxylamine succinate and at least about 80% of pyridoxine HCl dissolved within 140 minutes, as measured by USP Type II Apparatus at 100 rpm in change-over Media (for 2 hours in 900 ml of 0.1N HCl and then 1 hour in 900 ml at pH 6.5 phosphate buffer or pH 7.5 phosphate buffer).

The disintegrant-free delayed release pharmaceutical composition according to the present invention comprises doxylamine succinate and pyridoxine HCl along with at least one pharmaceutically acceptable excipient, and preferably is substantially free of lactose.

Preferably, the delayed release pharmaceutical composition comprising doxylamine succinate and pyridoxine HCl along with at least one pharmaceutically acceptable excipient selected from the group consisting of: binders, fillers, diluents, hydrophilic polymers, lubricants, glidants, surfactants, coating polymers and combinations thereof.

Also preferably, the filler and diluent is selected from the group consisting of: hydrophilic excipients or hydrophilic polymers, comprising one or more of mannitol, glucose, sorbitol, cellulose, calcium phosphate, starch, sugar and combinations thereof. More preferably, the filler and diluent is mannitol that is present in amount ranging from about 10% w/w to about 80% w/w of the total composition.

Preferably, the filler-diluent is selected from the group consisting of: cellulose, modified cellulose, sodium carboxymethyl cellulose, ethyl cellulose hydroxymethyl cellulose, cellulose acetate, hydroxypropylcellulose, microcrystalline cellulose, dibasic calcium phosphate, sucrose, corn starch, potato starch and combinations thereof. Preferably, the filer-diluent is dibasic calcium phosphate dihydrate that is present in amount ranging from about 1% w/w to about 25% w/w of the total composition. More preferably, that dibasic calcium phosphate dihydrate is present in amount ranging from about 1% w/w to about 20% w/w of the total composition.

In addition to the active ingredient, the pharmaceutical composition of the present invention contains pharmaceutically acceptable excipients, like binder which is selected from the group consisting of: cellulose or modified cellulose such as microcrystalline cellulose and cellulose ethers, hydroxypropyl cellulose (HPC), plant cellulose, sodium carboxymethyl cellulose, ethyl cellulose hydroxymethyl cellulose, polyvinylpyrrolidone, cellulose acetate, dibasic calcium phosphate, sucrose, glucose, mannitol, xylitol, sorbitol, starches and combinations thereof.

Preferably, the binder is hypromellose and is present in amount ranging from about 0.5% w/w to about 10% w/w of the total composition. More preferably the binder is hypromellose present in amount ranging from about 1% w/w to about 2% w/w of the total composition.

The compositions of the present invention may also comprise a lubricant. Preferably the lubricant is selected from the group consisting of: magnesium stearate, calcium stearate, zinc stearate, sodium stearate, stearic acid, aluminum stearate, leucine, glyceryl behenate, hydrogenated vegetable oil and combinations thereof. Preferably, the lubricant is magnesium stearate and is present in amount ranging from about 0.1% w/w to about 2% w/w of the total composition.

Preferably, the delayed release pharmaceutical composition comprises at least one enteric coating. The enteric coating comprises: an aqueous acrylic enteric system (for example, Acryl-eze® White 93O18359) that is present in amount ranging from about 2% w/w to about 12% w/w of the total composition, preferably from about 1% w/w to about 6% w/w of the total composition; an Antifoam® 1520 that is present in amount ranging from about 0.1% w/w to about 0.3% w/w of the total composition, and said enteric coated tablets are printed using Opacode Pink®.

The delayed release pharmaceutical composition according to the present invention is substantially free of lactose, microcrystalline cellulose, sodium croscarmelose and other disintegrants.

Oral dosage forms which may be employed with the present invention include granules, pellets in a capsule or in any other suitable solid form. Preferably, however the oral dosage form is a tablet.

According to the present invention, a disintegrant-free delayed release pharmaceutical composition for oral administration comprising: doxylamine succinate and pyridoxine HCl along with at least one pharmaceutically acceptable excipient, which provides an in vitro drug release profile of both pharmaceutically active ingredient as measured by USP Type II apparatus, at 100 rpm in 900 ml at pH 6.5 phosphate buffer as follows:
more than 50% of doxylamine succinate and more than 60% of pyridoxine HCl is released after 10 minutes;
about 80% of doxylamine succinate and about 80% of pyridoxine MCI is released after 20 minutes;
preferably, about 90% of doxylamine succinate and about 90% of pyridoxine HCl is released after 20 minutes.

Also, the present invention provides a disintegrant-free delayed release doxylamine succinate and pyridoxine HCl pharmaceutical composition, which provides an in vitro drug release profile of both pharmaceutically active ingredients as measured by USP Type II apparatus, at 100 rpm in 900 ml at pH 7.5 phosphate buffer as follows:
more than 50% of doxylamine succinate and more than 60% of pyridoxine HCl is released after 10 minutes;
preferably, about 80% of doxylamine succinate and about 80% of pyridoxine HCl is released after 10 minutes;
about 80% of doxylamine succinate and about 80% of pyridoxine HCl is released after 15 minutes;
preferably, about 90% of doxylamine succinate and about 90% of pyridoxine HCl is released after 15 minutes.

Preferably, the present invention provides a disintegrant-free delayed release pharmaceutical composition for oral administration comprising: doxylamine succinate and pyridoxine Ha along with at least one pharmaceutically acceptable excipient, wherein an in vitro dissolution profile of said pharmaceutical composition provides more than 50% of doxylamine succinate and more than 50% of pyridoxine HCl dissolved within 10 minutes, as measured by USP Type II Apparatus, at 100 rpm in 900 ml at pH 6.5 phosphate buffer.

Also preferably, the present invention provides a disintegrant-free delayed release pharmaceutical composition for oral administration comprising doxylamine succinate and pyridoxine HCl along with at least one pharmaceutically acceptable excipient, wherein an in vitro dissolution profile of said pharmaceutical composition provides of more than 50% of doxylamine succinate and more than 50% of pyridoxine HCl dissolved within 10 minutes, as measured by USP Type II Apparatus, at 100 rpm in 900 ml at pH 7.5 phosphate buffer.

More preferably, an in vitro dissolution profile of said pharmaceutical composition provides of more than 80% of the each active ingredient dissolved within 10 minutes, as measured by USP Type II Apparatus, at 100 rpm in 900 ml at pH 7.5 phosphate buffer.

Another object of present invention provides a disintegrant-free delayed release pharmaceutical composition for oral administration comprising:
a) at least one pharmaceutically active ingredient;
b) at least one filler;
c) at least one binder;
d) at least one lubricant; and
e) at least one enteric coating, wherein said composition provides an in vitro drug release profile of at least 80% of active ingredient dissolved within 20 minutes as measured by USP Type II apparatus, at 100 rpm in 900 ml at pH 6.5 phosphate buffer.

Preferably, said composition provides an in vitro drug release profile of about 95% of active ingredient dissolved within 20 minutes as measured by USP Type II apparatus, at 100 rpm in 900 ml at pH 6.5 phosphate buffer.

Preferably, said composition provides an in vitro drug release profile of about 95% of active ingredient dissolved within 20 minutes as measured by USP Type II apparatus, at 100 rpm in 900 ml at pH 7.5 phosphate buffer.

More preferably said composition provides an in vitro drug release profile of about 95% of active ingredient dissolved within 140 minutes as measured by USP Type II apparatus, at 100 rpm in change-over Media (for 2 hours in 900 ml of 0.1N HCl and then 1 hour in 900 ml at pH 6.5 phosphate buffer or pH 7.5 phosphate buffer).

Also preferably, the disintegrant-free delayed release pharmaceutical composition for oral administration comprising:
a) a core with at least one pharmaceutically active ingredient and with at least one pharmaceutically acceptable excipient, and
b) a coating which envelops the core and which comprises Acryl-eze White®, and Antifoam®, wherein said composition provides an in vitro drug release profile of about 80% of active ingredient dissolved within 20 minutes as measured by USP Type II apparatus, at 100 rpm in 900 ml at pH 6.5 phosphate buffer.

More preferably, said composition provides an in vitro drug release profile of about 95% of active ingredient dissolved within 20 minutes as measured by USP Type II apparatus, at 100 rpm in 900 ml at pH 6.5 phosphate buffer.

Also preferably, said composition provides an in vitro drug release profile of more than 80% of active ingredient dissolved within 140 minutes as measured by USP Type II apparatus, at 100 rpm in change-over Media (for 2 hours in 900 ml of 0.1N HCl and then 1 hour in 900 ml at pH 6.5 phosphate buffer or at pH 7.5 phosphate buffer).

Preferably, a disintegrant-free delayed release pharmaceutical composition for oral administration comprising: (a) doxylamine succinate and pyridoxine HCl as the active pharmaceutical ingredient, (b) mannitol as a filler-diluent, (c) dibasic calcium phosphate dehydrate as a filler-diluent, and (d) hypromellose as a binder, and (e) acrylic enteric polymer coating that comprising Acryl-eze White® and Antifoam®.

More preferably, a disintegrant-free delayed release pharmaceutical composition comprises:
a) about 5 to 10% w/w of doxylamine succinate;
b) about 5 to 10% w/w of pyridoxine HCl;
c) about 1 to 20% w/w of dibasic calcium phosphate dihydrate;
d) about 10 to 80% w/w of mannitol;
e) about 0.5 to 10% w/w of hypromellose;
f) about 0.1 to 2% w/w of magnesium stearate; and
   an enteric coating which envelops the core comprises:
g) about 1 to 12% w/w of Acryl-eze® (White 93O18359);
h) about 0.1 to 0.3% w/w of Atifoam®; and
j) optionally an Opacode®.

The delayed release pharmaceutical composition can be manufactured in accordance with usual techniques by direct compression or dry granulation method.

The present invention provides a process for manufacturing a disintegrant-free delayed release pharmaceutical composition comprising an inert core. The inert core comprises: about 5 to 10% w/w of doxylamine succinate; about 5 to 10% w/w of pyridoxine HCl; about 1 to 25% w/w of dibasic calcium phosphate dihydrate; about 10 to 80% w/w of mannitol; about 0.5 to 10% w/w of hypromellose; about 0.1 to 2% w/w of magnesium stearate; and an enteric coating that envelops the core. The enteric coating comprises: about 2 to 12% w/w of an acrylic enteric polymer (for example, Acryl-eze® (White 93O18359)); about 0.1 to 0.3% w/w of an anti-frothing agent (for example, Antifoam®); and h) the coding is optionally printed on with a colorant, such as Opacode®.

Preferably, the process is direct compression and comprises the following steps:
a) mixing hypromellose, doxylamine succinate, pyridoxine HCl and a portion of mannitol in a suitable blender;
b) passing the mixture of step (a) through a Comil equipped with a 0.024″R sieve at low speed with a round impeller;
c) mixing the mixture of step (b) and another portion of mannitol in a suitable blender;
d) passing the mixture of step (c) through a Comil equipped with a 0.032″R sieve at low speed with a round impeller;
e) mixing mixture of step (d) and dibasic calcium phosphate dihydrate, and the rest of the mannitol remaining in the formulation in a suitable blender;
f) passing the mixture of step (e) through a Comil equipped with a 0.032″R sieve at low speed with a round impeller;
g) re-introducing the blend from step (f) in a suitable bin blender, for 12 minutes at 14 rpm;
h) mixing magnesium stearate and a small portion of the blend of step (g) and disperse for 30 seconds;
i) passing the blend of step (h) through a Comil equipped with a 0.018″R sieve at low speed with a round impeller;
j) incorporating the sieved mixture from step (i) with the rest of the blend from step (g) in a suitable bin blender and mixing for 3 minutes at 14 rpm;
k) compressing obtained blend in step (j) using rotary tablet press;
l) coating obtained core tablets with Acryl-eze® and Antifoam®1520 coating dispersion, and optionally on each coated tablet is printed “P” logo with Opacode® Pink S-1-14022.

### EXAMPLES

The following example illustrates the preferred embodiment and various aspects of the present invention and is not to be considered as limiting the invention in any way.

### Example 1

### Formulation and Method of Producing a Disintegrant-Free Delayed Release Pharmaceutical Composition Containing Doxylamine Succinate and Pyridoxine HCl

The required quantity of Hypromellose, doxylamine succinate, pyridoxine HCl and a portion of mannitol ARE continuously mixed in a suitable blender, thereby forming mixture (#1).

Mixture (#1) is passed through a Comil equipped with a 0.024″R sieve at low speed with a round impeller, thereby forming mixture (#2).

Mixture (#2) is mined with another portion of mannitol in a suitable blender, thereby forming mixture (#3).

Mixture (#3) is passed through a Comil equipped with a 0.032″R sieve at low speed with a round impeller, thereby forming mixture (#4).

Mixture (#4) is mixed with the required quantity of dibasic calcium phosphate dihydrate, and the rest of the mannitol remaining in the formulation in a suitable blender, thereby forming mixture (#5).

Mixture (#5) passed through a Comil equipped with a 0.032″R sieve at low speed with a round impeller, thereby forming blend (#6).

Blend (#6) is re-introduced in a suitable bin blender, for 12 minutes at 14 rpm, thereby forming blend (#7).

The required quantity of magnesium stearate is mixed with a small portion of the blend of step (#7) and disperse for 30 seconds, thereby forming blend (#8).

Blend (#8) is passed through a Comil equipped with a 0.018″R sieve at low speed with a round impeller, thereby forming mixture (#9).

The sieved mixture from step (#9) is incorporated with the rest of blend (#7) in a suitable bin blender and mixed for 3 minutes at 14 rpm to form blend (#10);

Blend (#10) is compressed using rotary tablet press;

The core tablets obtained by this process are then coated with Acryl-eze® and Antifoam®1520 coating dispersion, and optionally on each coated tablet is printed a logo using a colorant, for example, a “P” logo with Opacode Wink S-1-14022.

The formulation of Example 1 is set out in Table I below.

TABLE I
The formulation of Example 1 for a delayed-release composition.

| | | | | % |
|---|---|---|---|---|
| No. | Ingredients | Function | mg/tablet | (w/w) |
| 1 | Doxylamine Succinate | API | 10.0 | 5.0 |
| 2 | Pyridoxine Hydrochloride | API | 10.0 | 5.0 |
| 3 | Dibasic Calcium | filler-diluent | 32.0 | 16.0 |
| | Phosphate Dihydrate | | | |
| 4 | Mannitol | filler-diluent | 141.5 | 70.75 |
| 5 | Hypromellose | binder | 4.0 | 2.0 |
| 6 | Magnesium stearate | lubricant | 2.5 | 1.25 |
| | Subtotal of core tablet | — | 200.0 | 100 |
| 7 | Acryl-eze White 93O18359 | coating | 11.43 | 5.71 |
| 8 | Antifoam 1520 | anti-frothing | 0.57 | 0.29 |
| | | agent | | |
| | Total of the coated tablet | | 212.0 | 106 |
| 9 | Opacode Pink S-1-14022 | colorant | q.s. | q.s. |

### Dissolution Data for Example 1

The pharmaceutical composition obtained from above mentioned Example 1 was subsequently tested for in vitro dissolution rate, measured by USP Type II Apparatus, using the following parameters:
Speed—100 rpm
Change-over Media—0.1N HCl and pH 6.5 or pH 7.5 phosphate buffer
Dissolution medium—900 ml
Temperature—37° C.
Time—2 hours at 0.1N HCl and then 1 hour at pH 6.5 or pH 7.5 phosphate buffer.

The dissolution of the tablet prepared according to Example 1 was compared to the dissolution of Dicletcin. These results are set out in Tables II and III below.

TABLE II
Dissolution Data at pH 6.5 Phosphate Buffer.
Drug release profiles with apparatus II at 100 rpm in change-over Media
(2 hours in 900 ml of 0.1N HCl and then 1 hour in 900 ml of pH 6.5
phosphate buffer)

| | Diclectin ® Lot#: 1150 | | Example 1 | |
|---|---|---|---|---|
| | Percentage | Percentage | Percentage | Percentage |
| | release for | release for | release for | release for |
| Time | doxylamine | pyridoxine | doxylamine | pyridoxine |
| (minutes) | Succinate (%) | HCl (%) | Succinate (%) | HCl (%) |
| 0 | 0 | 0 | 0 | 0 |
| 120 | 0 | 0 | 0 | 1 |
| 130 | 53 | 58 | 54 | 64 |
| 140 | 91 | 95 | 99 | 101 |
| 150 | 95 | 98 | 99 | 101 |
| 165 | 96 | 98 | 100 | 101 |
| 180 | 96 | 98 | 99 | 101 |

According to the present example, an in vitro dissolution profile of doxylamine succinate and pyridoxine HCl disintegrant-free delayed-release pharmaceutical composition provides about 80% of doxylamine succinate and about 80% of pyridoxine HCl dissolved within 140 minutes as measured by USP Type II apparatus, at 100 rpm in change-over media (for 2 hours in 900 ml of 0.1N HCl and then 1 hour in 900 ml of pH 6.5 phosphate buffer).

Preferably, the pharmaceutical composition of the present invention provides an in vitro dissolution profile of at least about 50% of doxylamine succinate and about 50% of pyridoxine HCl dissolved within 10 minutes, as measured by USP Type II Apparatus, at 100 rpm in 900 ml at pH 6.5 phosphate buffer (for example, see the 130 minute mark in Table II).

More preferably, the pharmaceutical composition of the present invention provides an in vitro dissolution profile of about 90% of each active ingredient dissolved within 20 minutes as measured by USP Type II apparatus, at 100 rpm in 900 ml at pH 6.5 phosphate buffer (for example, see the 140 minute mark in Table II).

TABLE III
Dissolution Data at pH 7.5 Phosphate Buffer.
Drug release profiles with apparatus II at 100 rpm in change-over media
(2 hours in 900 ml of 0.1N HCl and then 1 hour in 900 ml of pH 7.5
phosphate buffer)

| | Diclectin ® Lot#: 1173V | | Example 1 | |
|---|---|---|---|---|
| | Percentage | Percentage | Percentage | Percentage |
| | release for | release for | release for | release for |
| Time | doxylamine | pyridoxine | doxylamine | pyridoxine |
| (minutes) | Succinate (%) | HCl (%) | Succinate (%) | HCl (%) |
| 0 | 0 | 0 | 0 | 0 |
| 120 | 0 | 0 | 0 | 0 |
| 130 | 95 | 95 | 90 | 93 |
| 135 | 98 | 97 | 98 | 98 |
| 150 | 99 | 99 | 99 | 99 |
| 165 | 99 | 99 | 99 | 99 |
| 180 | 99 | 99 | 99 | 99 |

According to the present example, an in vitro dissolution profile of doxylamine succinate and pyridoxine HCl disintegrant-free delayed-release pharmaceutical composition provides at least 80% of doxylamine succinate and at least 80% pyridoxine NCI dissolved within 130 minutes as measured by USP Type II apparatus, at 100 rpm in change-over Media (for 2 hours in 900 ml of 0.1N HCl and then 1 hour in 900 ml at pH 7.5 phosphate buffer).

Preferably, the pharmaceutical composition of the present invention provides an in vitro dissolution profile of at least about 80% of doxylamine succinate and at least about 80% pyridoxine HCl dissolved within 10 minutes as measured by USP Type II apparatus, at 100 rpm in Media in 900 ml at pH 75 phosphate buffer (for example, see the 130 minute mark in Table III).

More preferably, the pharmaceutical composition of the present invention provides an in vitro dissolution profile of about 85% of doxylamine succinate and about 85% pyridoxine HCl dissolved within 15 minutes, as measured by USP Type II Apparatus, at 100 rpm in 900 ml at pH 7.5 phosphate buffer (for example, see the 140 minute mark in Table III).

More preferably, the in vitro dissolution profile of said composition provides about 90% of doxylamine succinate and about 90% pyridoxine HCl dissolved within 20 minutes as measured by USP Type II apparatus, at 100 rpm in 900 ml of pH 7.5 phosphate buffer (for example, see the 140 minute mark in Table III).

1. A disintegrant-free delayed release pharmaceutical composition for oral administration comprising:
a core;
an enteric coating;
wherein said core comprises:
at least one pharmaceutically active ingredient, and
at least one pharmaceutically acceptable excipient,
wherein said composition provides an in vitro drug release profile of about 95% of active ingredient dissolved within 20 minutes as measured by USP Type II apparatus at 100 rpm in 900 ml at pH 6.5 phosphate buffer.

2. The pharmaceutical composition according to claim 1, wherein the pharmaceutically active ingredient is doxylamine succinate, pyridoxine hydrochloride or a combination thereof.

3. The pharmaceutical composition according to claim 2, wherein the composition comprises 10 mg of doxylamine succinate and 10 mg of pyridoxine hydrochloride.

4. A disintegrant-free delayed release pharmaceutical composition for oral administration comprising doxylamine succinate and pyridoxine HCl along with at least one pharmaceutically acceptable excipient,
wherein the pharmaceutical composition provides an in vitro dissolution profile of about 80% of each active ingredient dissolved within 20 minutes, as measured by USP Type II Apparatus at 100 rpm in 900 ml at pH 6.5 phosphate buffer.

5. A disintegrant-free delayed release pharmaceutical composition for oral administration comprising doxylamine succinate and pyridoxine HCl along with at least one pharmaceutically acceptable excipient,
wherein the pharmaceutical composition provides an in vitro dissolution profile of about 80% for each of the each active ingredients dissolved within 20 minutes, as measured by USP Type II Apparatus at 100 rpm in 900 ml at pH 7.5 phosphate buffer.

6. A disintegrant-free delayed release pharmaceutical composition for oral administration comprising doxylamine succinate and pyridoxine HCl along with at least one pharmaceutically acceptable excipient,
wherein the pharmaceutical composition is substantially free of lactose; and
wherein the pharmaceutical composition provides an in vitro dissolution profile of about 80% for each of the each active ingredients dissolved within 20 minutes, as measured by USP Type II Apparatus at 100 rpm in 900 ml at pH 6.5 phosphate buffer.

7. The pharmaceutical composition according to claim 1, wherein said composition comprises doxylamine succinate and pyridoxine HCl along with at least one pharmaceutically acceptable excipient selected from the group consisting of binders, fillers, diluents, hydrophilic polymers, lubricants, glidants, surfactants, coating polymers and combinations thereof.

8. The pharmaceutical composition according to claim 7, wherein the filler and diluent is selected from the group consisting of: hydrophilic excipients or hydrophilic polymers, comprising one or more of mannitol, glucose, sorbitol, cellulose, calcium phosphate, starch, sugar and combinations thereof.

9. The pharmaceutical composition according to claim 7, wherein the filler and diluent is mannitol.

10. The pharmaceutical composition according to claim 9, wherein the mannitol is present in amount ranging from about 10% w/w to about 80% w/w of the total composition.

11. The pharmaceutical composition according to claim 7, wherein the filler is selected from the group consisting of cellulose, modified cellulose, sodium carboxymethyl cellulose, ethyl cellulose hydroxymethyl cellulose, cellulose acetate, hydroxypropylcellulose, dibasic calcium phosphate, sucrose, corn starch, potato starch and combinations thereof.

12. The pharmaceutical composition according to claim 8, wherein the filler and diluent is dibasic calcium phosphate dihydrate.

13. The pharmaceutical composition according to claim 12, wherein dibasic calcium phosphate dihydrate is present in amount ranging from about 1% w/w to about 25% w/w of the total composition.

14. The pharmaceutical composition according to claim 7, wherein the binder is selected from the group consisting of cellulose or modified cellulose, hydroxypropyl cellulose, plant cellulose, sodium carboxymethyl cellulose, ethyl cellulose hydroxymethyl cellulose, hydroxypropyl methylcellulose, hypromellose, polyvinylpyrrotidone, cellulose acetate, dibasic calcium phosphate, sucrose, glucose, mannitol, xylitol, sorbitol, starches and combinations thereof.

15. The pharmaceutical composition according to claim 14, wherein the binder is hypromellose.

16. The pharmaceutical composition according to claim 15, wherein the hypromellose is present in amount ranging from about 0.5% w/w to about 10% w/w of the total composition.

17. The pharmaceutical composition according to claim 7, wherein the lubricant is selected from the group consisting of: magnesium stearate, calcium stearate, zinc stearate, sodium stearate, stearic acid, aluminum stearate, leucine, glyceryl behenate, hydrogenated vegetable oil and combinations thereof.

18. The delayed release pharmaceutical composition according to claim 17, wherein the lubricant is magnesium stearate and is present in amount ranging from about 0.1% w/w to about 2% w/w of the total composition.

19. The pharmaceutical composition for oral administration according to claim 1, wherein said formulation is substantially free of lactose and disintegrants.

20. The composition according to claim 1, wherein the enteric coating comprises an aqueous acrylic enteric coating; wherein the aqueous acrylic enteric coating is present in amount ranging from about 2% w/w to about 12% w/w of the total composition.

21. The pharmaceutical composition according to claim 1, wherein the enteric coating further comprises an anti-frothing agent present in amount ranging from about 0.1% w/w to about 0.3% w/w of the total composition.

22. The pharmaceutical composition according to claim 1, wherein said enteric coated composition is printed using colorant.

23. The pharmaceutical composition according to claim 1, wherein the pharmaceutical composition is a tablet.

24. The pharmaceutical composition according to claim 1, wherein the composition provides an in vitro dissolution profile of more than about 50% of doxylamine succinate and more than about 50% of pyridoxine HCl dissolved within 10 minutes, as measured by USP Type II Apparatus, at 100 rpm in 900 ml at pH 6.5 phosphate buffer.

25. The pharmaceutical composition according to claim 1, wherein the composition provides an in vitro dissolution profile of more than about 50% of doxylamine succinate and more than about 50% of pyridoxine HCl dissolved within 10 minutes, as measured by USP Type II Apparatus, at 100 rpm in 900 ml at pH 7.5 phosphate buffer.

26. A disintegrant-free delayed release pharmaceutical composition for oral administration comprising:
a) at least one pharmaceutically active ingredient;
b) at least one filler;
c) at least one binder;
d) at least one lubricant; and
e) at least one enteric coating,
wherein said composition provides an in vitro drug release profile of about 95% of active ingredient dissolved within 20 minutes as measured by USP Type II apparatus, at 100 rpm in 900 ml at pH 6.5 phosphate buffer.

27. A disintegrant-free delayed release pharmaceutical composition for oral administration comprising:
a) at least one pharmaceutically active ingredient;
b) at least one filler;
c) at least one binder;
d) at least one lubricant; and
e) at least one enteric coating,
wherein said composition provides an in vitro drug release profile of about 95% of active ingredient dissolved within 20 minutes as measured by USP Type II apparatus, at 100 rpm in 900 ml at pH 7.5 phosphate buffer.

28. The pharmaceutical composition according to claim 26, wherein:
(a) the at least one pharmaceutically active ingredient comprises doxylamine succinate and pyridoxine HCl;
(b) the filler comprises mannitol and dibasic calcium phosphate dehydrate;
(d) the binder comprises hypromellose; and
(e) the enteric coating comprises an acrylic enteric polymer coating.

29. A disintegrant-free delayed release pharmaceutical composition for oral administration comprising:
a) a core with at least one pharmaceutically active ingredient and with at least one pharmaceutically acceptable excipient;
b) a coating which envelops the core, the coating comprising an acrylic enteric polymer coating and an anti-frothing agent,
wherein said composition provides an in vitro drug release profile of about 80% of active ingredient dissolved within 20 minutes as measured by USP Type II apparatus, at 100 rpm in 900 ml at pH 6.5 phosphate buffer.

30. A disintegrant-free delayed release pharmaceutical composition for oral administration comprising:
a) a core with at least one pharmaceutically active ingredient and with at least one pharmaceutically acceptable excipient;
b) a coating which envelops the core, the coating comprising an acrylic enteric polymer coating and an anti-frothing agent,
wherein said composition provides an in vitro drug release profile of about 80% of active ingredient dissolved within 20 minutes as measured by USP Type II apparatus, at 100 rpm in 900 ml at pH 7.5 phosphate buffer.

31. The delayed release pharmaceutical composition of claim 1, wherein said composition is prepared by direct compression or dry granulation.

32. Use of a delayed release pharmaceutical composition according to claim 1, for the treatment of nausea and vomiting during pregnancy.

33. The use according to claim 32, wherein said delayed release pharmaceutical composition provides an in vitro dissolution profile of about 80% of the each active ingredient dissolved within 15 minutes, which is substantially equivalent to the dissolution of Diclectin®.
